(19) United States
(12) Patent Application Publication
(10) Pub. No.: US 2007/0299325 A1
(43) Pub. Date: Dec. 27, 2007

(54) Physiological status monitoring system

(76) Inventors: Brian Farrell, Quincy, MA (US); Richard Streeter, Winchester, MA (US); Jeremy Bowman, Arlington, MA (US); David McDonald, Medway, MA (US); Paul Nahass, Cambridge, MA (US)

Correspondence Address: IANDIORIO & TESKA;INTELLECTUAL PROPERTY LAW ATTORNEYS, 260 BEAR HILL ROAD, WALTHAM, MA, 02451-1018, US

(21) Appl. No.: 11/807,449

(22) Filed: May 29, 2007

Related U.S. Application Data
(63) Continuation-in-part of application No. 10/922,336, filed on Aug. 20, 2004.

(51) Int. Cl. A61B 5/00

(52) U.S. Cl. 600/301

(57) ABSTRACT

A physiological status monitoring system includes a shirt and a stretchable circumferential band attached to the shirt. The stretchable band includes a respiration detector subsystem and signal transmission conductors. One or more sensors on the band are electrically connected to a signal transmission conductor. At least one sensor has an exposed electrode inside the shirt. The system includes a cover over the band and/or the one or more sensors. A connection subsystem on the band is electrically connected to the respiration detector subsystem and the signal transmission conductors and includes signal traces therefrom to a first connector accessible from outside the shirt. An electronics module is releasably attached to the shirt and includes a second connector which mates with the first connector. The electronics module includes a processing system and a transmitter. The remote display unit includes a receiver, a display, and a processing system.

### RELATED APPLICATIONS

This application is a continuation-in-part application of U.S. patent application Ser. No. 10/922,336, filed Aug. 20, 2004, which is incorporated herein by reference.

### FIELD OF THE INVENTION

The subject invention relates to a physiological monitoring system able to monitor and report a person's vital signs such as ECG, respiration, and the like.

### BACKGROUND OF THE INVENTION

Different versions of physiological monitoring systems, some integrated to a certain extent in clothing, have been proposed. The idea is to be able to monitor the vital signs (e.g., heart rate, respiration rate, and the like) as a subject performs his, her, or its normal activities or duties. Such a system could be used by military personnel, fire fighters and police officers, athletes, patients, and animals.

Vivometrics (see www.vivometrics.com) offers the “Life Shirt” as but one example of a physiological monitoring shirt-based system. See also, for example, U.S. Pat. Nos. 6,047,203; 6,474,367; D451,604; 6,605,038; and 6,494,829 incorporated herein by this reference.

To date, however, no commercial product seems to meet the needs of the marketplace. That is, some physiological monitoring systems are not comfortable to wear. Others are difficult to use. Some require preparation prior to and/or after donning the garment. Some include discrete wires which must be routed and/or connected each time the garment is worn. Some include electrodes which must be secured to the person's body and/or must be used in connection with a conductive gel. Some physiological monitoring garments are simply not aesthetically pleasing. Others interfere with the activities of and duties carried out by the wearer. Finally, special precautions must be taken in order to clean certain garments equipped with physiological monitoring sensors and electrodes.

### SUMMARY OF THE INVENTION

It is therefore an object of this invention to provide a new physiological monitoring system.

It is a further object of this invention to provide such a system which is preferably garment based and comfortable to wear.

It is a further object of this invention to provide such a system which is easy to use.

It is a further object of this invention to provide such a system which requires minimal or no preparation prior to or after donning the garment.

It is a further object of this invention to provide such a system which does not include any wires which must be connected or routed by the wearer.

It is a further object of this invention to provide such a system which does not require the user to secure electrodes to her body or to use any conductive gels.

It is a further object of this invention to provide such a system which is aesthetically pleasing.

It is a further object of this invention to provide such a system which does not interfere with the activities of or duties carried out by the wearer.

It is a further object of this invention to provide such a system which can be more easily cleaned.

The subject invention results from the realization that a better physiological monitoring garment such as a shirt includes a stretchable circumferential band including both a respiration sensing subsystem and signal/power transmission conductors for one or more other sensors also on the band and an easily removable electronics module electrically connected to the respiration sensing subsystem and the sensors via the band for transmitting signals representing the wearer's respiration rate and/or depth, heart rate, and the like but without any loose wires, separate electrodes, and therefore comfortable to wear, easy to use, and easily washed or cleaned.

The subject invention, however, in other embodiments, need not achieve all these objectives and the claims hereof should not be limited to structures or methods capable of achieving these objectives.

The subject invention features a physiological status monitoring system including a shirt and a stretchable circumferential band attached to the shirt. The stretchable circumferential band includes a respiration detector subsystem integral with the band and signal transmission conductors also integral with the band and in a flexible configuration. One or more sensors on the band are each electrically connected to a signal transmission conductor. At least one sensor has an electrode exposed inside the shirt. There is a cover over the band and/or the one or more sensors. A connection subsystem on the band is electrically connected to the respiration detector subsystem and the signal transmission conductors and includes signal traces therefrom to a first connector accessible from outside the shirt. An electronics module is releasably attached to the shirt and includes a second connector which mates with the first connector. The electronics module includes a processing system for processing signals from the respiration detector subsystem and the signal transmission conductors, and a transmitter for wirelessly transmitting the processed signals. A remote display unit includes a receiver which receives the transmitted processed signals, a display, and a processing system for displaying the received processed signals on the display. The electronics module may further include a receiver for receiving communications from the remote display unit.

The shirt may be made of Lycra material. The shirt may include fabric having fibers of variable loft and thickness. The respiration detector subsystem may include in-plane circumferential conductors integral with the stretchable band, and the in-plane conductors may have a sinusoidal configuration. In one variation respiration detector subsystem includes a pair of adjacent in-plane nested circumferential conductors integral with the stretchable band, which may be sinusoidal, triangle wave, zig-zag or other configuration. In such a variation the electronics module includes a circuit which detects changes in capacitance as the adjacent nested circumferential conductors move away from and towards each other as the stretchable band expands and contracts. In an alternative variation the electronics module includes a circuit which detects changes in inductance as circumferential conductors move as the stretchable band expands and contracts. The signal transmission conductors are also typically circumferential and may be sinusoidal or other suitable configuration.

The exposed electrode may be made of conductive fabric. One or more foam layers behind the conductive fabric promote contact of the conductive fabric with skin. A fabric over and behind the one or more foam layers is attached to the stretchable band and the shirt. Preferably, the one or more foam layers are water impenetrable to promote the conductivity of the conductive fabric. A conductive fastener extends through the conductive fabric and is connected to a conductor coupled to a signal transmission conductor in the band. One sensor may include a thermistor coupled to the band and having a conductor coupled to a signal transmission conductor. A side of the thermistor not in contact with a patient's body may be insulated. Typically, the cover is an outer cover and is made of fabric, which may be the same fabric as the shirt fabric.

The connection subsystem may include an insulation displacement connector, or a circuit board. The circuit board may be rigid or may be a flex circuit. A typical connector is a Lemo connector. Another typical connector is a pin connector. The connection subsystem may be encapsulated by a sealant.

The transmitter may be configured according to the Bluetooth or another standard and preferred remote display unit may be a hand held electronic device, such as a personal digital assistant (PDA). The system may further include a portal such as a website accessible over a network responsive to the remote display unit to display and log the processed signals. The electronics module may further include a GPS or other position detection subsystem, and/or a motion detector, such as an accelerometer. In one configuration the GPS or other position detection subsystem is separate from the electronics module, such as a stand alone GPS module, and in communication with the electronics module wirelessly. The electronics module may also include other sensors, such as a pulse oximeter (Sₚ0₂) or core body temperature sensor, or these sensors may be separate from the electronics module and in communication with the electronics module wirelessly. The electronics module may include a display for displaying the processed signals.

Hook and loop fasteners such as VELCRO® on the shirt and on the electronics module releasably attach the electronics module to the shirt. Other examples may include snap connectors for releasably attaching the electronics module to the shirt, or magnetic connectors for releasably attaching the electronics module to the shirt. The magnetic connectors may also provide electrical connectivity between the shirt and the electronics module. The processing system of the electronics module or the remote display unit may include a rules set including a rule in which signals from the respiration detector subsystem are not transmitted or an alert signal is sent if they indicate a breathing rate higher than possible by a subject wearing the shirt. The one or more sensors on the band may include electrocardiogram sensors located on opposite sides of the band and positioned to form a line across a wearer's heart.

In one embodiment the shirt is made of loose fitting material and includes an inner liner attached to the loose fitting material. In one variation, the stretchable circumferential band may be included in the inner liner. The loose fitting material may include at least one opening therein for releasable attachment of the electronics module to the shirt. In one example the inner liner extends to each edge of the loose fitting material. In another example, the inner liner extends from select edges of the loose fitting material to the stretchable material. In a further example, the inner liner is attached to the loose fitting material by connecting material segments.

The subject invention also features a physiological status monitoring system including a garment and a stretchable band attached to or integral with the garment. The stretchable band includes conductors integral with the band forming a respiration detector, and at least one signal transmission conductor also integral with the band. At least one sensor is electrically connected to the signal transmission conductor and an electronics module is responsive to the conductors and the signal transmission conductor. In one variation the sensor includes a thermistor coupled to the band and having a conductor coupled to a signal transmission conductor. A side of the thermistor not in contract with a wearer is typically insulated. Electrocardiogram sensors on the stretchable band may also be included and located on opposite sides of the band and positioned to form a line across a wearer's heart. The electronics module typically includes a circuit which detects changes in impedance as geometry of the conductors changes as the stretchable band expands and contracts, and a transmitter responsive to the circuit and to signals transmitted from the sensor for wirelessly transmitting respiration and sensor signals.

There may be an outside cover over the band. The system may further include a connection subsystem on the band electrically connected to the conductors and to the signal transmission conductor and including signal traces therefrom to a first connector accessible from outside the garment. The connection system may be an insulated displacement connector, or in another example, a circuit board. The circuit board may be rigid, or a flex circuit. The first connector may be a Lemo connector or a pin connector. The electronics module typically includes a second connector which mates with the first connector, and further includes a processing system for processing signals from the circuit and the signal transmission conductor. In one configuration the conductors are sinusoidal, and the at least one signal transmission conductor is sinusoidal. The system may further include a pair of adjacent nested conductors. The system also typically includes a remote display unit comprising a receiver which receives signals from the transmitter, a display, and a processing system for displaying the received signals on the display. The electronics module may include a receiver responsive to the remote display unit and to signals transmitted from the remote display unit for receiving communication from the remote display unit. The garment may be made of Lycra material, and include fabric having fibers of variable loft and thickness.

In one example, the band is circumferential and the conductors are in-plane. The sensors may include an exposed electrode made of conductive fabric. In one aspect there are one or more foam layers behind the conductive fabric to promote contact of the conductive fabric with a wearer's skin, and fabric over and behind the one or more foam layers and attached to the stretchable band. In one configuration the one or more foam layers are water impenetrable to promote conductivity of the conductive fabric. A conductive fastener through the conductive fabric may be included which is connected to a conductor coupled to a signal transmission conductor in the band.

In one configuration the electronics module may also include a position detection subsystem, which may be a GPS subsystem. Alternatively the position detection subsystem may be separate from but in communication with the electronics module. The electronics module may also include a motion detector such as an accelerometer, and/or a disply for displaying respiration and sensor signals.

In one embodiment the physiological monitoring system includes one of a hook and loop fastener on the garment and one of the hook and loop fastener on the electronics module for releasably attaching the electronics module to the garment. In another embodiment snap connectors releasably attach the electronics module to the garment. In yet a further embodiment magnetic connectors releasably attach the electronics module to the garment, and the magnetic connectors may further provide electrical connectivity between the garment and the electronics module. A processing system including a rules set including a rule in which signals from the respiration detector are not transmitted if they indicate a breathing rate higher than possible by a subject wearing the garment may be added to the physiological monitoring system.

In one aspect of the subject invention the garment is made of loose fitting material and includes an inner liner attached to the loose fitting material. In one variation, the stretchable band may be included in the inner liner. The loose fitting material may include at least one opening therein for releasable attachment of the electronics module to the garment. The inner liner may extend to each edge of the loose fitting material. Alternatively, the inner liner may extend from selected edges of the loose fitting material to the stretchable circumferential band. In another variation, the inner liner is attached to the loose fitting material via connecting material segments.

The subject invention also features a physiological status monitoring system including a stretchable band including a respiration detector subsystem integral with the band, and signal transmission conductors also integral with the band and in a flexible configuration. One or more sensors are diposed on the band and electrically connected to a signal transmission conductor, at least one sensor having an exposed electrode. A connection subsystem on the band is electrically connected to the respiration detector subsystem and the signal transmission conductors and including signal traces therefrom to a first connector. An electronics module with a second connector mates with the first connector, the electronics module including a processing system for processing signals from the respiration detector subsystem and the signal transmission conductors, and a transmitter for wirelessly transmitting the processed signals.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic front view of one example of a physiological monitoring shirt in accordance with the subject invention;

FIG. 2 is a rear view of the shirt shown in FIG. 1;

FIG. 3 is a schematic front view of one example of a remote display unit which interfaces with the electronics module shown in FIG. 1 for displaying the shirt wearer's physiological parameters;

FIG. 4 is a schematic front view of the inside of the shirt shown in FIG. 1 where an exposed ECG electrode is shown;

FIG. 5 is another schematic view of the inside of the shirt shown in FIG. 1 where another ECG electrode is shown and the outline of thermistor is also shown;

FIG. 6 is a schematic front top view of one embodiment of the stretchable circumferential band integrated into the shirt shown in FIGS. 1 and 2;

FIG. 7A is a highly schematic depiction showing conductors in the stretchable band shown in FIG. 6 when the band is in its relaxed state;

FIG. 7B is a highly schematic view similar to FIG. 7A except now the distance between the conductors in the band has changed because the band is in its expanded state;

FIG. 7C is an example of a plot of capacitance and time which shows respiration indicators in accordance with one aspect of the subject invention;

FIG. 8A is a schematic view showing the rear portion of one example of an ECG electrode in accordance with the subject invention electrically attached to a signal transmission conductor in the stretchable band;

FIG. 8B is a schematic three-dimensional top view of the ECG electrode shown in FIG. 8A;

FIG. 9 is a schematic exploded view of the ECG electrode shown in FIGS. 8A-8B;

FIG. 10A is a highly schematic view showing the rear portion of another example of an ECG electrode in accordance with the subject invention electrically attached to a signal transmission conductor in the stretchable band;

FIG. 10B is a schematic view of the ECG electrode shown in FIG. 10A;

FIG. 11 is a schematic three-dimensional view of one example of a thermistor useful in accordance with the subject invention the outline of which is shown in FIG. 5;

FIG. 12 is a schematic three-dimensional exploded view showing the primary components associated with the thermistor shown in FIG. 11;

FIG. 13 is another schematic three-dimensional top view of an example of a stretchable circumferential band of the subject invention now including a connection subsystem with a connector attached thereto in a sealed configuration on the band;

FIG. 14 is a schematic three-dimensional top view of one embodiment of a circuit board for use as a connection subsystem;

FIGS. 15A-15C are schematic three-dimensional views showing one example of how a connection subsystem and connector shown in FIG. 14 are rendered water proof in accordance with the subject invention;

FIG. 16A is another schematic front view of a physiological monitoring shirt in accordance with the subject invention showing one way of attaching the electronics module to the shirt in accordance with the present invention;

FIG. 16B is a schematic view of one way to attach a connection subsystem to the circumferential stretch band;

FIG. 16C is a schematic front view of another way of attaching the electronics module to the shirt in accordance with the present invention;

FIG. 16D is a more detailed schematic front perspective view of the connection subsystem shown in FIG. 16C;

FIG. 16E is a schematic front view of a further way of attaching the electronics module to the shirt in accordance with the present invention;

FIG. 17 is a schematic block diagram showing the primary components associated with a circuit board shown in FIGS. 14 and 16A;

FIG. 18 is a schematic block diagram showing the primary components typically associated with the electronics module shown in FIGS. 1 and 16A;

FIG. 19 is a block diagram showing one example of the primary components associated with the shirt and processing system or circuitry of the electronics module shown in FIG. 18;

FIG. 20 is a schematic block diagram showing the primary components typically associated with the display unit of FIG. 3;

FIG. 21 is a schematic front view of another example of a physiological monitoring shirt in accordance with the subject invention;

FIG. 22 is a schematic inside view of the shirt of FIG. 21;

FIG. 23 is a schematic partial inside view of the shirt of FIG. 21 including a partial cutaway view of a stretchable circumferential band in accordance with one aspect of the subject invention;

FIG. 24 is a schematic partially cutaway view of the shirt of FIG. 21;

FIG. 25A is a schematic partial view of the inside of a further example of a physiological monitoring shirt in accordance with the subject invention;

FIG. 25B is a schematic enlarged view of one portion of the inside of the shirt of FIG. 25A;

FIG. 26 is a schematic partially cutaway view of another example of a physiological monitoring shirt in accordance with the subject invention;

FIG. 27 is a schematic front view of one example of a physiological monitoring shirt including means for closing the shirt against the body of a wearer in accordance with one aspect of the subject invention; and

FIG. 28 is a schematic partial view of one example of a closable opening in a physiological monitoring shirt in accordance with one aspect of the subject invention; and

FIG. 29 is a schematic partial view of the opening shown in FIG. 28 in a closed configuration.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

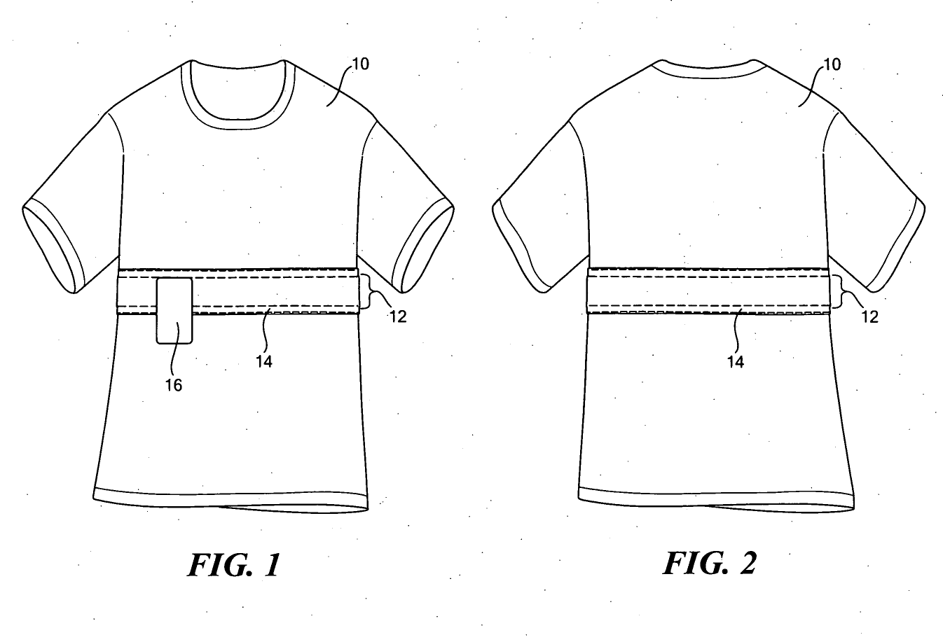

FIGS. 1-2 show one example of one version of a physiological status monitoring system in accordance with this invention. Easily washable garment or shirt 10 can be made of any fabric (e.g., cotton) but in this example is typically made of a “compression” fabric often including Lycra material (e.g., the POLARTEC® POWER STRETCH® material available from Malden Mills), although this is not a necessary limitation as discussed below. For additional comfort, moisture management and the like shirt 10 may include fabric fibers of variable loft, thickness or density placed to coincide with preferred body locations where desired. Sewn or bonded to this or any conventional shirt, in one example on the outside thereof, is a stretchable circumferential band the outline of which is shown in FIG. 1 at 12. In one configuration such as the configuration shown in FIG. 1, the stretchable circumferential band is integrated into the shirt. In other embodiments, discussed below, the shirt includes an inner liner or shell and the stretchable circumferential band is integrated into the liner of the shirt.

The result is a shirt free of any atypical seams or the like. The band includes an integrated respiration detection subsystem, sensors, signal transmission conductors for the sensors, and a connection subsystem such as a circuit board.

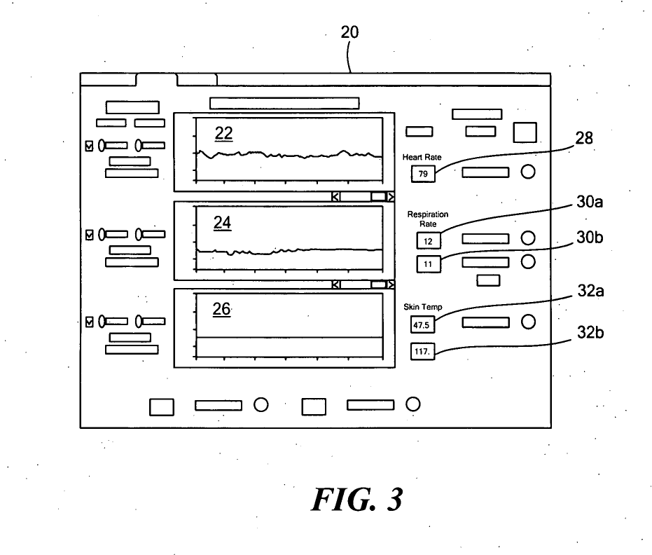

Cover 14, FIG. 1 also made of compression material is typically included, and in one variation is disposed on the outer side of circumferential band 12. Cover 14 is sewn, bonded or otherwise attached over the circumferential band and/or the sensors. Electronics module 16 is releasably attached to shirt 10 and is connected to the circuit board on the band. This electronics module wirelessly transmits respiration and other (e.g., ECG) physiological status signals to remote display unit 20, FIG. 3 where the wearer's heart rate is displayed on display 22, respiration is displayed on display 24, and skin temperature is displayed on display 26. Numerical readouts are also provided as shown at 28 (heart rate), 30a and 30b (e.g. respiration rate and depth), and 32a and 32b (skin temperature).

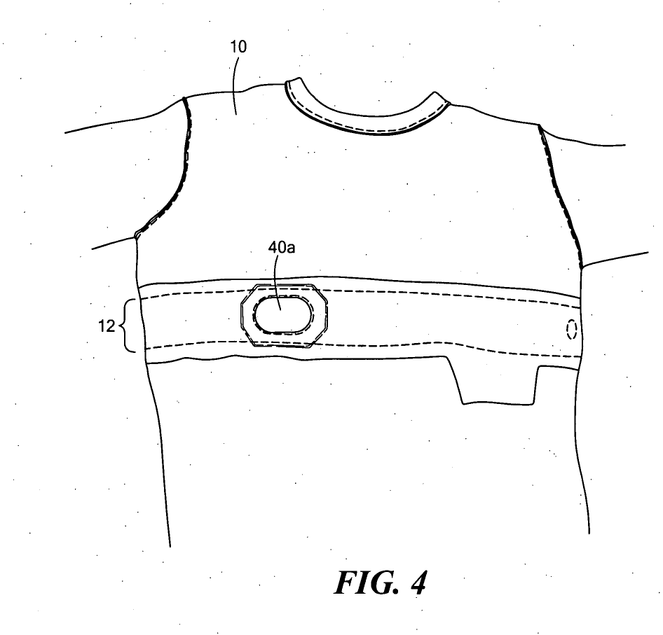

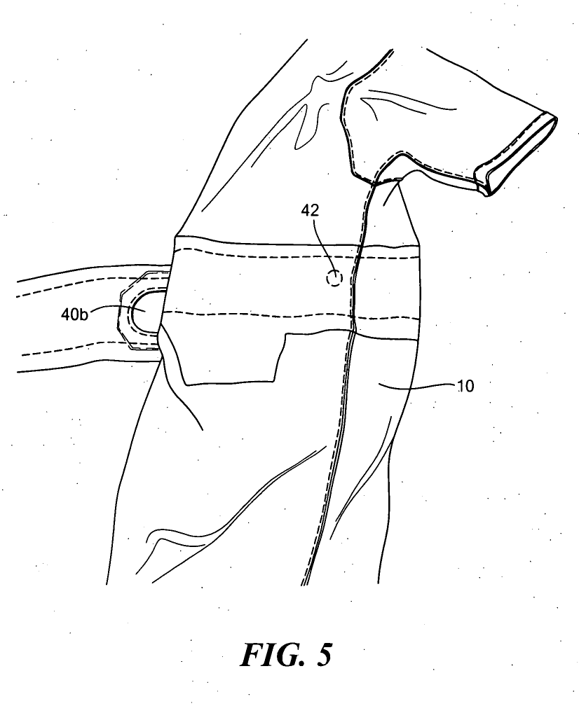

FIG. 4 shows the inside of one embodiment of shirt 10 and again the outline of the circumferential band can be seen at 12. FIG. 4 also shows one exposed ECG electrode 40a inside the shirt for monitoring the wearer's heart rate. FIG. 5 shows another exposed ECG electrode 40b and the outline of thermistor 42 for monitoring skin temperature. Other sensors may be added and may be integrated with the band or connected to it. Examples include thoracic bioimpedance sensors or biomechanical sensors connected to the signal transmission elements of the band.

Note the lack of any loose wires inside or outside the shirt. Other than electrodes 40a and 40b, and the thermistor, only shirt material touches the wearer's skin. Except for electronics module 16, FIG. 1 and the slight outline of the band, shirt 10 looks just like a normal shirt. Shirt 10 is thus comfortable, aesthetically pleasing, quickly donnable and doffable, and easy to use. It can be worn under other clothing, it is easily cleaned, it wicks away body perspiration, and it does not interfere with the activities of or duties carried out by the wearer. Physiological parameters measured are more accurate because the portion of the shirt including the circumferential band can hold sensors in more intimate contact with the wearer's body. Also, the sensors are located away from the module so that they do not move with movement of the wearer, resulting in further increased accuracy of measurements.

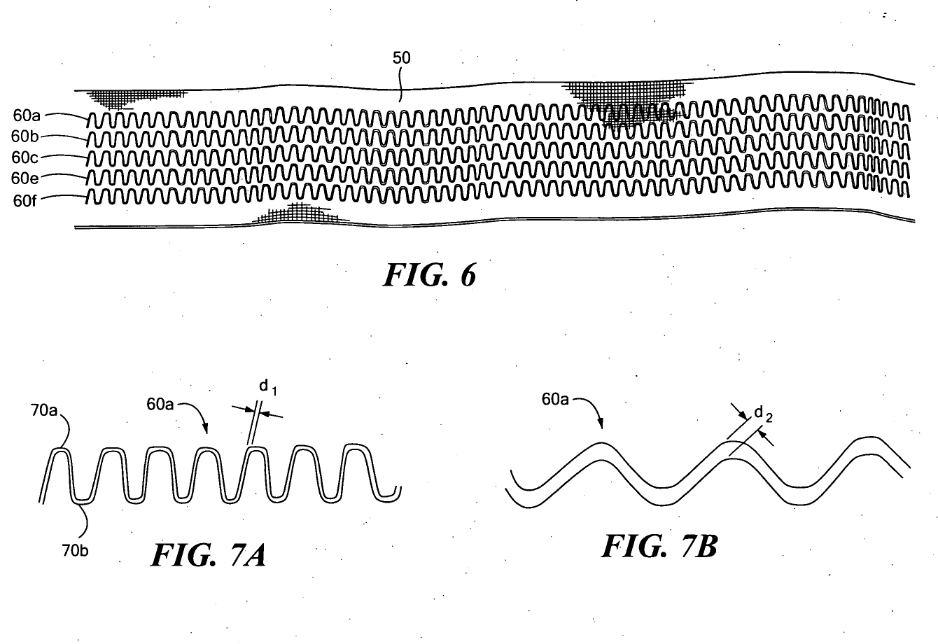

Stretchable circumferential band 50 is shown alone in FIG. 6. Integrated with the fabric of band 50 are conductors (typically insulated wires) in a flexible configuration. In one non-limiting example the conductors include in-plane nested pairs as shown at 60a-60e. The conductors need not be in nested pairs, but may be singularly arranged, and in either arrangement the conductors—whether singular or in nested pairs—may be sinusoidal as shown, or any other suitable configuration such as triangle wave or zig-zag (not shown). In this way changes in impedance, e.g. inductance or capacitance, can be measured as the conductors move as the stretchable band expands or contracts, and the conductors can be used to form the respiration detection subsystem. When the band is relaxed because the wearer has exhaled, the geometry of the wires changes. When the band is stretched because the wearer has inhaled, the geometry of the wires again changes. In this way, by configuring band 50, FIG. 6 to be circumferential about the wearer's chest and snug thereabout in the relaxed configuration, when the wearer breathes, conductors in the band can be used as a respiration detector.

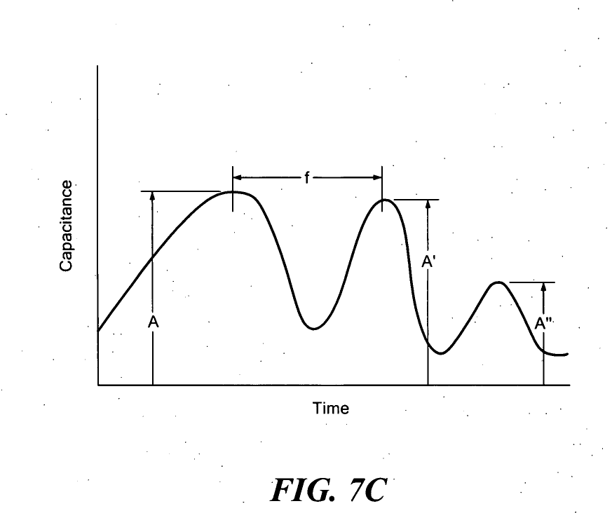

In one variation, a conductor pair 60a is shown more clearly in FIGS. 7A-7B. When the band is relaxed because the wearer has exhaled, the distance between wires 70a and 70b is d₁, FIG. 7A. When the band is stretched because the wearer has inhaled, the distance between wires 70a and 70b l is d₂, FIG. 7B. In this example, a nested conductor pair in the band is used as a respiration detector.

Electronics module 16, FIG. 1 is electrically connected to the conductors, such as flexible wires, and includes a circuit which detects changes in impedance, such as changes in inductance or changes in capacitance as desired for a particular application. As noted above, impedance will change as the conductors move, particularly as the geometry of a circumferential conductor changes. That change in impedance is thus indicative of respiration rate, indicating frequency of breaths taken by the wearer, as well as the depth or volume of each breath. In the configuration in which changes in capacitance are detected, electronics module 16 is electronically connected to the two wires 70a and 70b, and the circuit detects changes in capacitance as adjacent nested circumferential conductor pairs move away and towards each other as the stretchable band expands and contracts as shown in FIGS. 7A-7B. In a plot of capacitance and time, FIG. 7C, peak to peak distance f is indicative of breathing rate or frequency. Amplitudes A, A′ . . . indicate the depth of each breath, which can be important in the overall evaluation of the physical condition of the wearer. These indications can be processed and transmitted to display unit 20, FIG. 3 for display and/or read out. Loop inductance (see U.S. Pat. Nos. 6,783,498 and 6,413,225 incorporated herein by this reference) and other impedance-based respiration sensing techniques may also be used.

Other conductor pairs can also be used for sensing respiration but typically at least a few conductors are reserved for signal transmission from a sensor such as ECG electrodes 40a and 40b and thermistor 42, FIGS. 4-5 to electronics module 16, FIG. 1 and possibly between electronics module 16 and these and other sensors which may be included on or electrically connected to the band. In one configuration, ECG sensors 40a and 40b are located on opposite sides of the band such that one ECG sensor 40a is located in the front and one ECG sensor 40b is located on the back of the wearer of the shirt, the sensors 40a, 40b positioned to form points on a line directly across the wearer's heart. This positioning together with the stability and the sensors intimate body contact provided by the ECG sensors' attachment to the circumferential band of the shirt, provides an improved electrical signal indicative of heartbeat.

One method for integrating wire conductors into a ribbon is disclosed in U.S. Pat. No. 6,727,197 incorporated herein. See also copending application Ser. No. 10/922,336 filed Aug. 20, 2004 also incorporated herein by this reference.

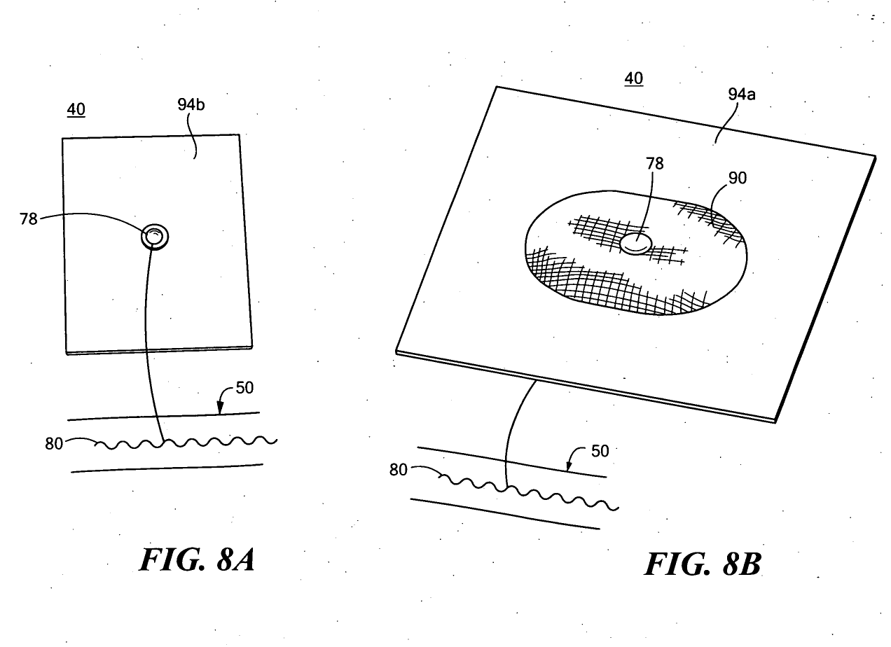

Connection of the various sensors to the band can vary. In one example, the back of snap 78, FIG. 8A of ECG electrode 40 is connected (e.g., soldered) to a wire 80 designated as a signal transmission conductor in band 50. ECG electrode 40 is typically sewn to both band 50 and shirt 10 as shown in FIG. 4, although this is not a necessary limitation, and ECG electrode 40 may be bonded or otherwise attached to the band and shirt, or to the band and an inner liner portion of the shirt, the latter discussed in more detail below.

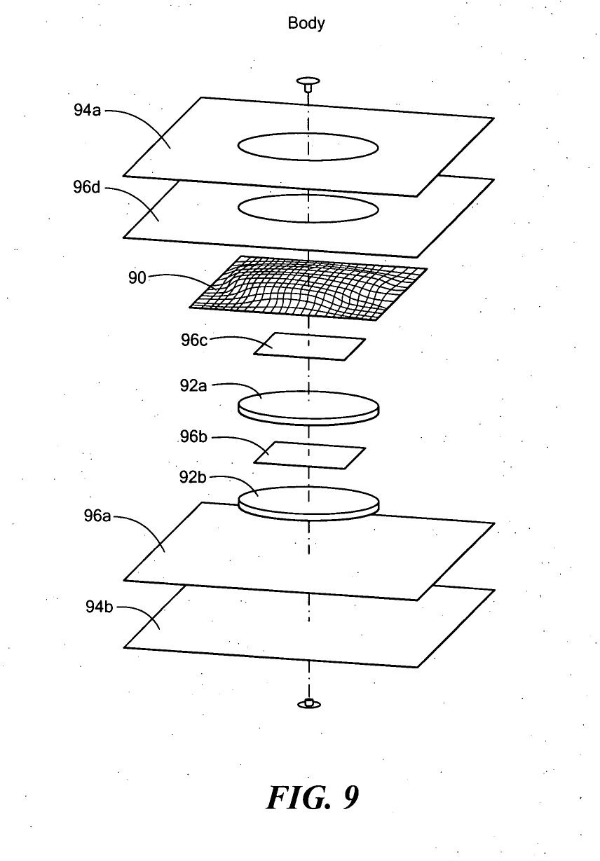

FIG. 8B shows the exposed conductive fabric portion 90 of electrode 40 which is pressed against the wearer's skin. A typical ECG electrode configuration includes water impermeable or resistant foam layers 92a and 92b, FIG. 9 behind conductive fabric layer 90 to promote contact of conductive fabric layer 90 with the skin and to improve the conductivity of fabric layer 90 as the wearer of shirt perspires.

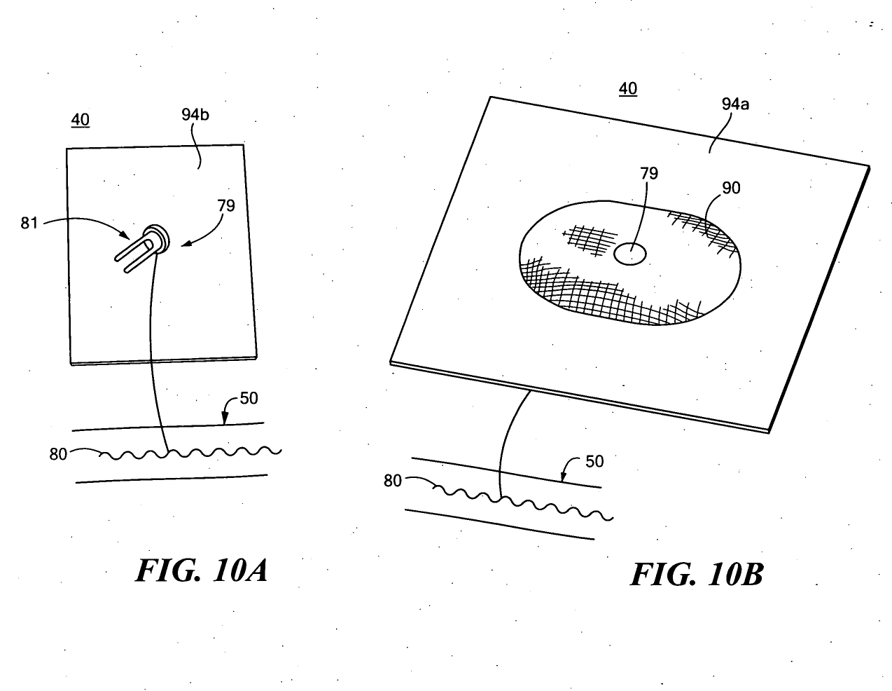

Fabric cover 94a and 94b sandwich the conductive fabric layer and the foam layers together as a unit as shown in FIGS. 8A-8B for ease of assembly when cover layers 94a and 94b are sewn to each other and to the stretchable band and the shirt or part thereof, after a portion of one signal transmission conductor is freed from the band, stripped of insulation, and soldered directly to or via a dog leg to ECG lead rivet 78, FIGS. 8A-8B fastened through all the layers of the electrode. Cloth and/or pressure sensitive adhesive layers 96a-d, FIG. 9 assist in securing fabric cover layers 94a and 94b, foam layers 92a and 92b, and electrode fabric layer 90 together. Suitable materials for fabric layer 90 include silver coated nylon, or stainless steel fibers woven with nylon threads. In another configuration, a low-profile insulation displacement connector (IDC) 79, FIGS. 10A and 10B is used to establish an electrical connection to wire 90. The “v-shaped” metal bracket portion 81, FIG. 10A would cut through insulation e.g. on wire 80 to establish the electrical connection.

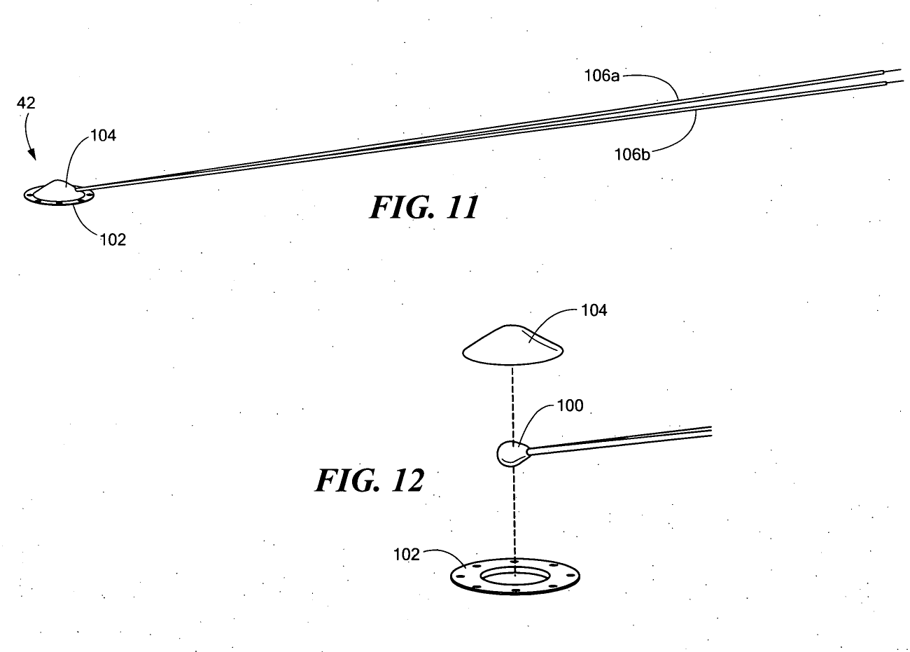

Thermistor 42 is shown in more detail in FIGS. 11-12. Thermistor element 100, FIG. 12 is held in place with respect to thermistor plate 102 via thermal epoxy 104 and wires 106a and 106b are connected to respective signal transmission conductors (e.g. nested pair 60e, FIG. 6) in the stretch band. Plate 102 is attached (e.g., stitched) to the stretch band so the raised side of plate 102 faces inward towards the wearer's body. In one configuration, the side of thermistor 42 not in contact with the wearer's body is insulated for improved performance.

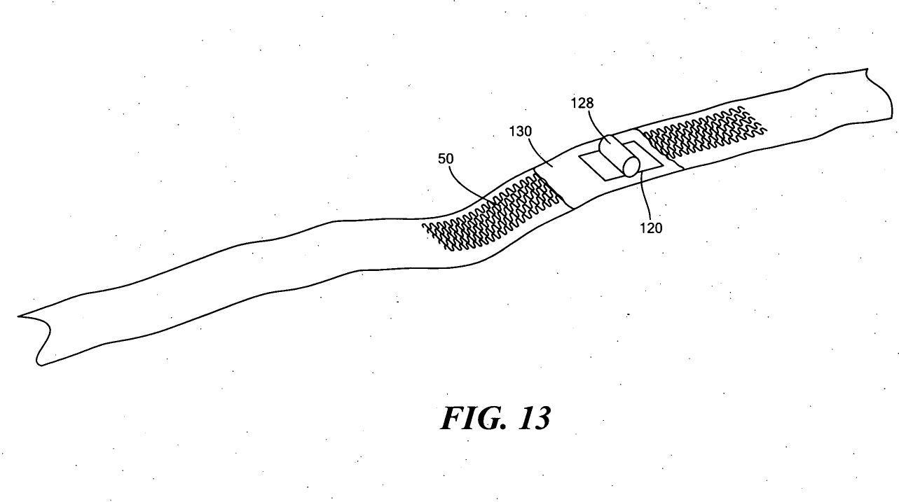

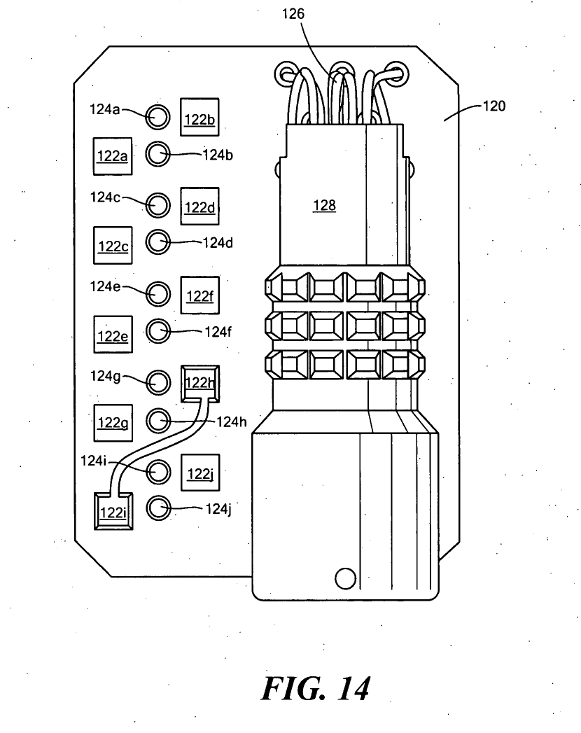

FIG. 13 shows one version of small (approximately 2″×2″) circuit board 120 on band 50, which serves as a connection subsystem to band 50. The purpose of circuit board 120 is to provide a connection point for the nested conductors in the band with electronics module 16, FIG. 1. Circuit board 120, FIG. 14 is rigid in this example and includes pads 122a-122j proximate holes 124a-j. The conductors of the band are threaded through holes 124a-j and soldered to pads 122a-122j. Conductive traces in circuit board 120 (not shown) then route electrical signals from pads 122a-122j to wires 126 of Lemo connector 128. This connector is accessible from outside the shirt, in one example through the front of the shirt, and is connected to a connector associated with electronics module 16, FIG. 1.

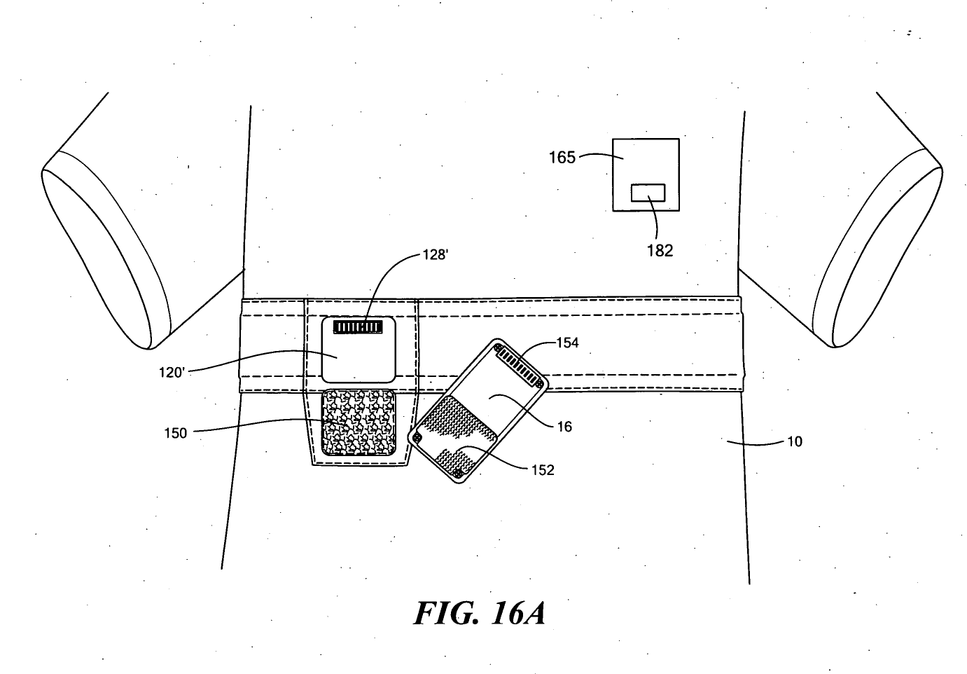

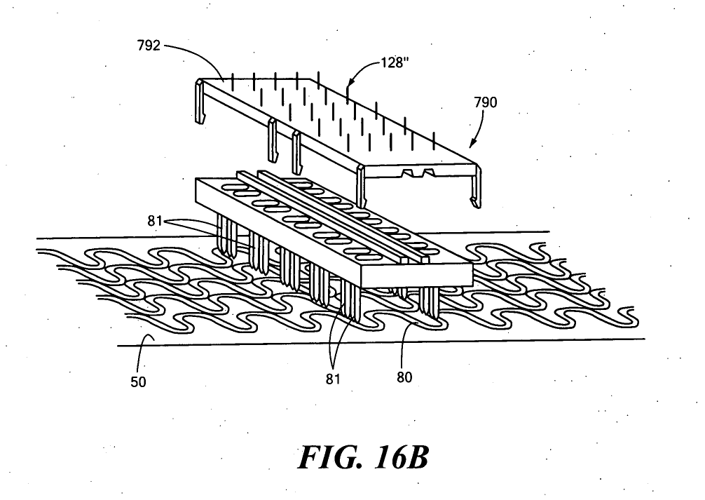

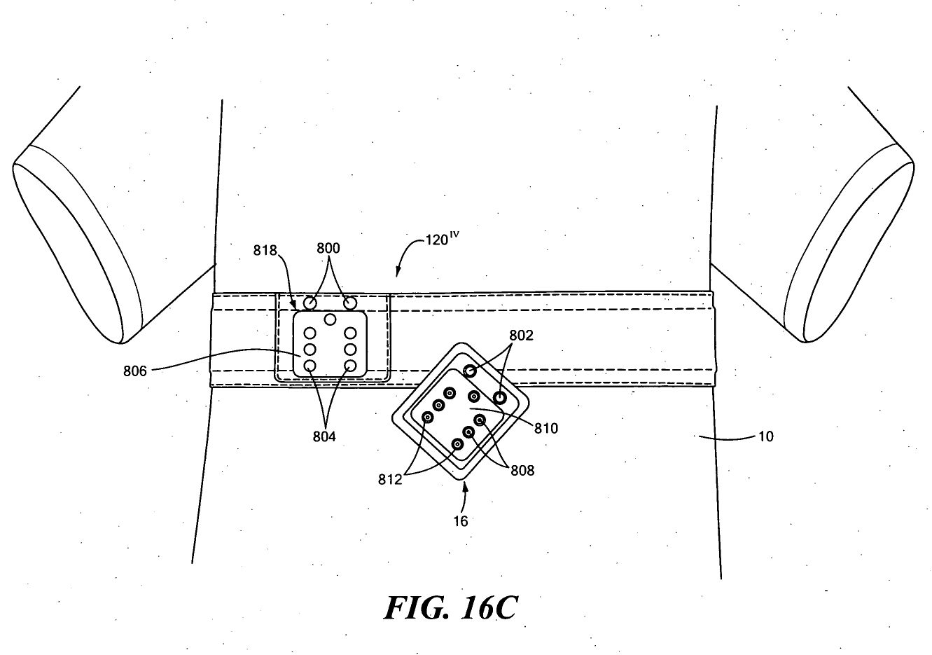

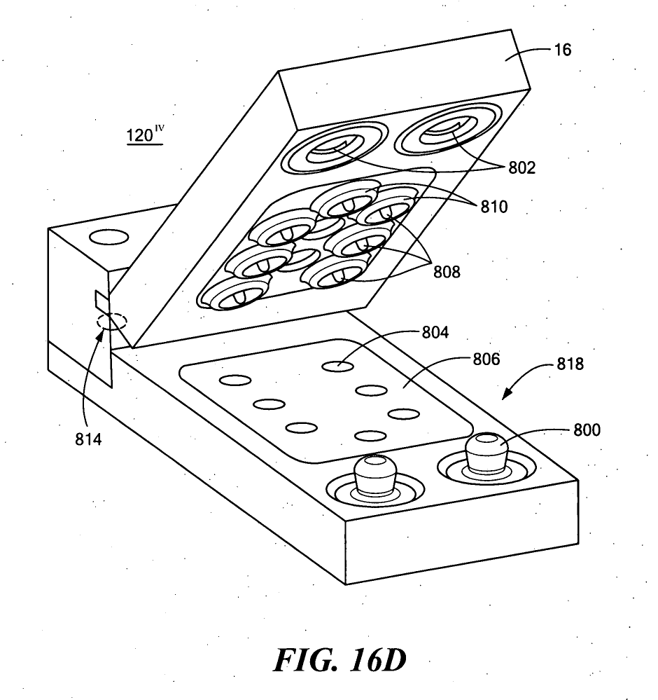

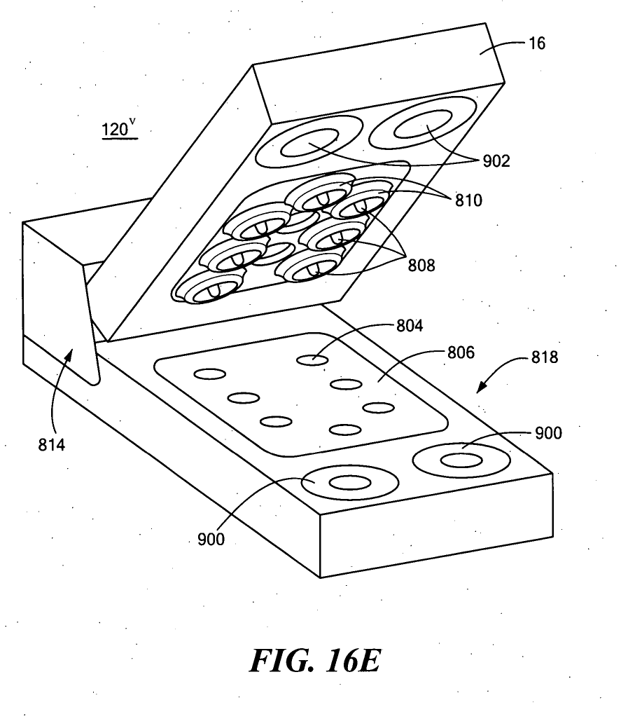

Circuit board 120 may also be a flex circuit as disclosed in U.S. Pat. No. 6,729,025 incorporated herein by this reference. Also, different connectors may be used including a pin connector (discussed below with respect to FIG. 16) or an insulation displacement connector (IDC) and the like. Such connectors are also accessible from outside the shirt, also typically but not necessarily through the front of the shirt.

In any embodiment, the connection subsystem or circuit board 120 is typically rendered water proof as shown in FIG. 13 by silicone, epoxy or another encapsulant 130 about both the connection subsystem or circuit board 120 and connector 128.

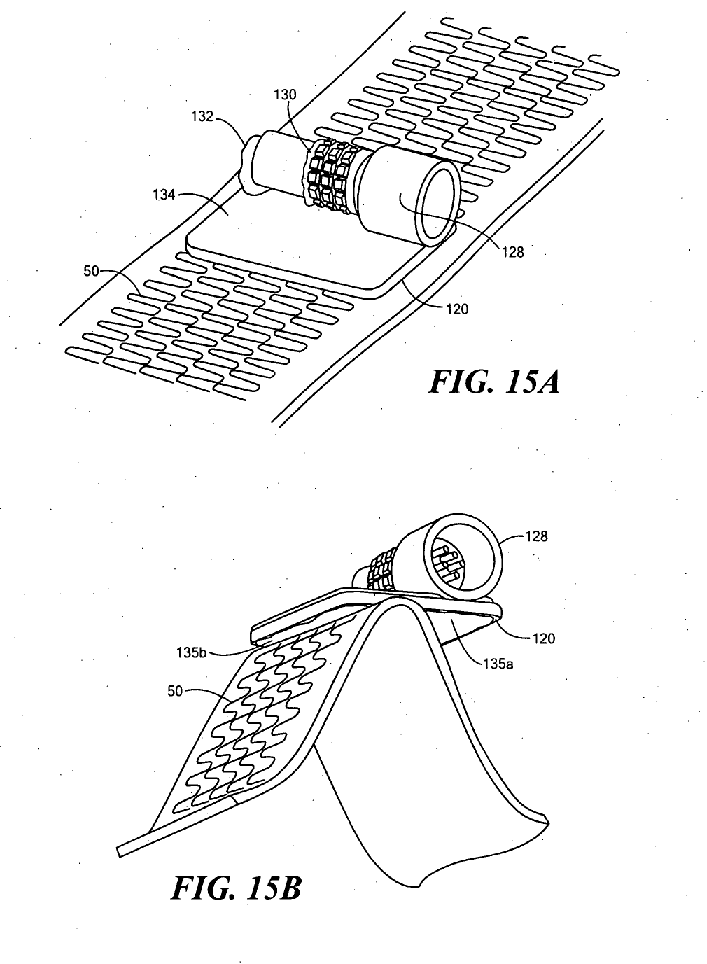

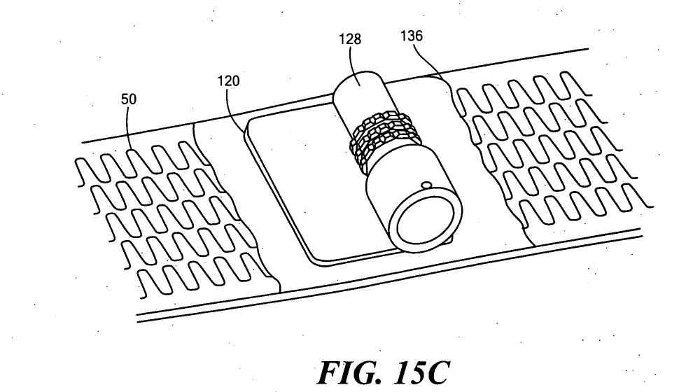

FIGS. 15A-15C show one method of encapsulation after the conductors of band 50 are soldered to circuit board 120 and connector 128 is secured to circuit board 120 using a cyanoacrylate epoxy. In FIG. 15A, a bead of epoxy 130 is placed over the threads of Lemo connector 128, and epoxy is injected into the back end of Lemo connector 128 to completely cover all the wires as shown at 132. All the wires and any exposed conductor surfaces are coated with an epoxy as shown at 134. Next, silicone is smeared on the bottom surface of printed circuit board 120 as shown at 135a and 135b, FIG. 15B. Next, silicone is smeared over the entire border of circuit board 120 as shown in FIG. 15C at 136, and as shown in the completed form in FIG. 13.

FIG. 16A shows a different kind of connection subsystem 120′, such as a circuit board with pin connectors 128′ and one of a hook and loop fastener 150 such as a VELCRO® patch on shirt 10 proximate circuit board 120′. Electronics module 16 includes the other of a hook and loop fastener 152 such as a VELCRO® patch for releasably securing electronics module 16 to shirt 10 as shown in FIG. 1. Connectors 154 of module 16 mate with pin connectors 128′ of circuit board 120′. In yet another variation, connection subsystem 120 includes insulation displacement connector (IDC) 790, FIG. 16B including multiple v-shaped metal bracket portions 81 which mate or connect with wires 80 in band 50. Top portion 792 of IDC 790 may contain elements e.g. pads or pin connectors 128″ which connect with connectors 154, FIG. 16A on module 16.

FIG. 16C shows a different kind of connection subsystem 120^{IV} for connecting electronics module 16 to shirt 10. This embodiment includes snap connectors 800 for releasably securing electronics module 16 to shirt 10. Snap connectors 800 mate with corresponding snap connector holes 802 in module 16. Pads 804 on circuit board 806 engage with corresponding spring fingers 808 in module 16 to establish electrical connections. In one example, sealing portion 810 includes an o-ring 812 for each of the spring fingers 808 for more effective sealing to prevent water and/or moisture from penetrating to spring fingers 808 and pads 804. A more detailed view of the connection subsystem of FIG. 16C is shown in FIG. 16D. In this example, module 16 is detachable at point 814 and includes snap connector holes 802, spring fingers 808, and o-rings 810. Portion 818 of connection subsystem 120^{IV} on shirt 10 includes snap connectors 800, pads 804 and circuit board 806. This is not a necessary limitation, however, and module 16 may include snap connectors 800, pads 804 and circuit board 806 while portion 818 includes snap connector holes 802, spring fingers 808 and o-rings 810.

In another embodiment, connection subsystem 120^{V} includes magnetic connectors 900, FIG. 16E may be used for releasably securing electronics module 16 to shirt 10 as shown in FIG. 1. Corresponding magnetic connectors 902 of opposite polarity provide the attracting force to releasably secure module 16 to shirt 10. Magnetic connectors 900 and 902 are typically made of metal, and in one example provide an electrical connection between module 16 and shirt 10 in addition to releasably securing module 16 to shirt 10. In this example, the remaining elements are the same as those shown in FIGS. 16C and 16D and operate in similar fashion to those in connection subsystem 120^{IV}, and are similarly interchangeable, although these are not necessary limitations of the invention. Additionally, in one configuration, sealing portion 810 and o-rings 810 include silicon, and pads 804 are made of copper, although any suitable material as desired for a particular application may be utilized.

A preferred connection subsystem/connector combination is low profile and small in size for comfort and the electronics module is releasable therefrom in order to clean shirt 10. It will be understood that the connection subsystem in accordance with the embodiments of the subject invention also ultimately connects the electronics module to the signal transmission conductors from the sensors and with the respiration subsystem in the stretchable circumferential band. The connection subsystem may also include a position detection system such as a GPS chip, one or more accelerometers or a gyroscope, and possibly other circuitry for providing signals to the remote display unit data indicating the wearer's position, movement, vital signs and the like. See e.g. http://www.trakus.com/technology.asp and http://www.phatrat.com both incorporated herein by this reference.

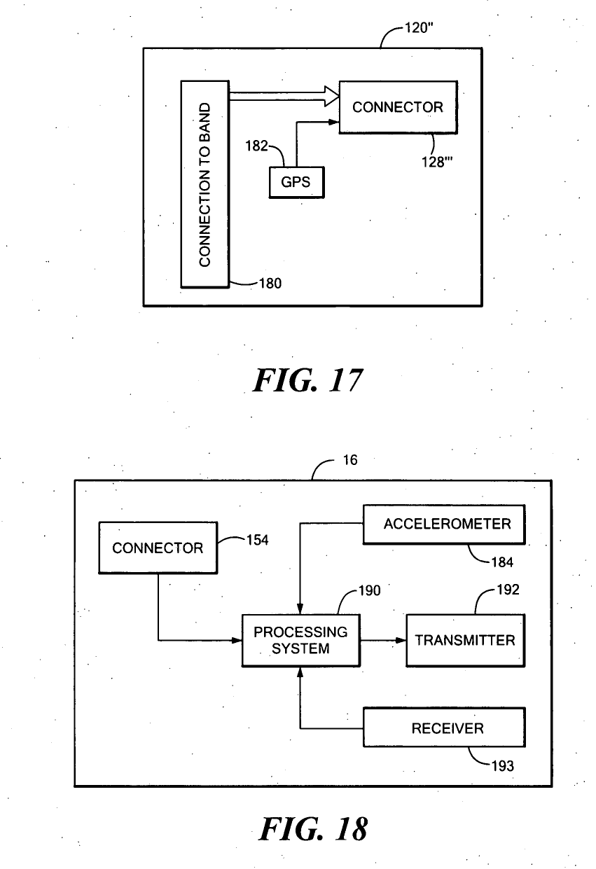

Connection subsystem 120″, FIG. 17 such as a circuit board in one example, includes a connection to the band conductors as shown at 180 and position detection subsystem 182, such as a GPS unit, connected to connector 128′″. Micro-electromechanical systems may be used. Connector 154 of electronics module 16, FIG. 18 releasably mates with connector 128′″, FIG. 17. Electronics module 16, FIG. 18 includes processing system 190 or circuitry (e.g., a microprocessor or microcontroller) which processes the signals received from the respiration detection conductor pair(s) of the band and the other conductors in the band serving as signal transmission conductors and connected to sensors such as the ECG electrodes and thermistor sensor. Transmitter 192 wirelessly transmits these processed signals to remote display unit 22, FIG. 3. An accelerometer unit 184 may be included in electronics module 16. With respect to connection subsystem 120″, FIG. 17, certain circuitry of components or functionality may, however, be present on electronics module 16, FIG. 18 and vice versa. Also, in one variation position detection subsystem 182, FIG. 17, e.g. a GPS unit, may be a separate unit. For example, position detection subsystem 182 may include a stand alone module 165, FIG. 16A which wirelessly communicates with electronics module 16. The electronics module may also include other sensors, such as a pulse oximeter (Sₚ0₂) on core body temperature sensor, or these sensors may be separate from the electronics module and in communication with the electronics module wirelessly.

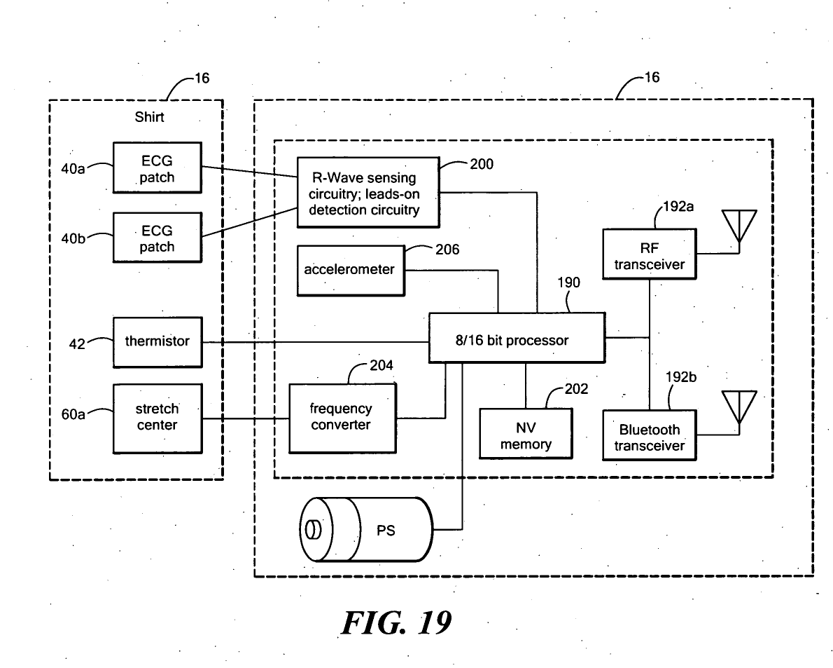

FIG. 19 shows in more detail the signal processing circuitry of the electronics module. The R-wave sensing circuitry and leads-on detection circuitry 200 receives and conditions the signals from ECG electrodes 40a and 40b to be processed by processor 190 which can store computed values in non-volatile memory 202. Signals from impedance stretch sensor wires, arranged singularly in the example of changes in inductance, or nested wire pairs 60a in the example of changes in capacitance, are converted to a frequency signal and received by frequency converter 204. This signal is also provided to processor 190. Signals from thermistor 42 and accelerometer 206 are also provided to processor 190. Processor 190 provides its output via RF transceiver 192a and/or Bluetooth transceiver 192b or similar transceiver system.

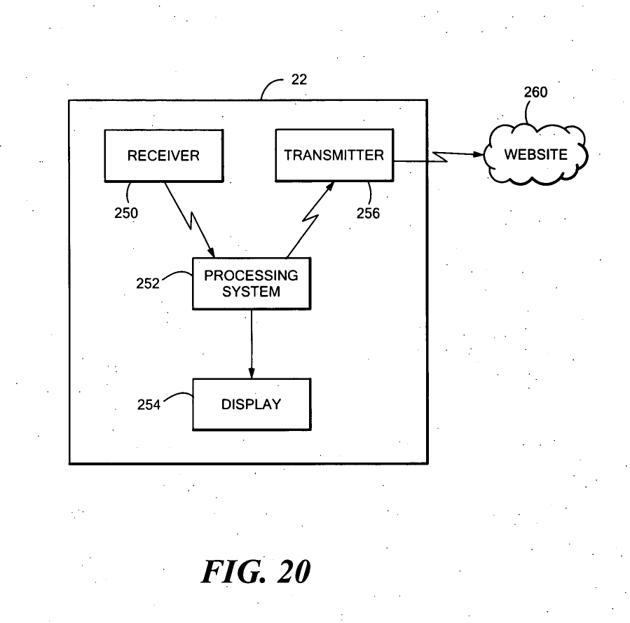

FIG. 20 shows the basic architecture of remote display unit 22, FIG. 3. Remote display unit 22 includes receiver 250 which receives the signals transmitted by the transmitter of electronics module 16, FIG. 1. Those signals are then processed by processing system 252, FIG. 20 for display on display 254 which, as shown in FIG. 3 may includes several individual displays. Remote display unit 22 may be a specially designed unit or, alternately, the transmitter of the electronics module can be configured according to the Bluetooth standard or some other standard in which case display unit 22 can be a hand held electronic device such as a personal digital assistant. Software can then be loaded into the personal digital assistant in order to provide a read out of the sensor and electrode signals as shown in FIG. 3. Also, display unit 22 can include transmitter 256 to transmit the signals to a portal such as website 260 where the signals can be viewed or logged for future reference and/or comparison. Electronics module 16, FIG. 18 can also include receiver 193 in which case bi-directional communications could be established between electronics module 16 and remote display unit 22, FIG. 20. This can be useful, for example, to query the electronics module to send additional data to the transmitter/display, or to alert the wearer of any situation.

Processor 190, FIG. 19 of electronics module 16 or processing system 252, FIG. 20 of remote display unit 22 may be programmed with various rules to recognize and respond to information which deviates too far from expected parameters, in order to provide for better system performance. One example is to filter out signals which are likely the result of body movement as opposed to breathing. For example, if the frequency of the signal provided to processor 190, FIG. 19 by frequency converter 204 indicates a breathing rate higher than is possible by the subject, processor 190 is programmed to e.g. send an alert signal or send no signal, rather than transmit the aberrant signal.

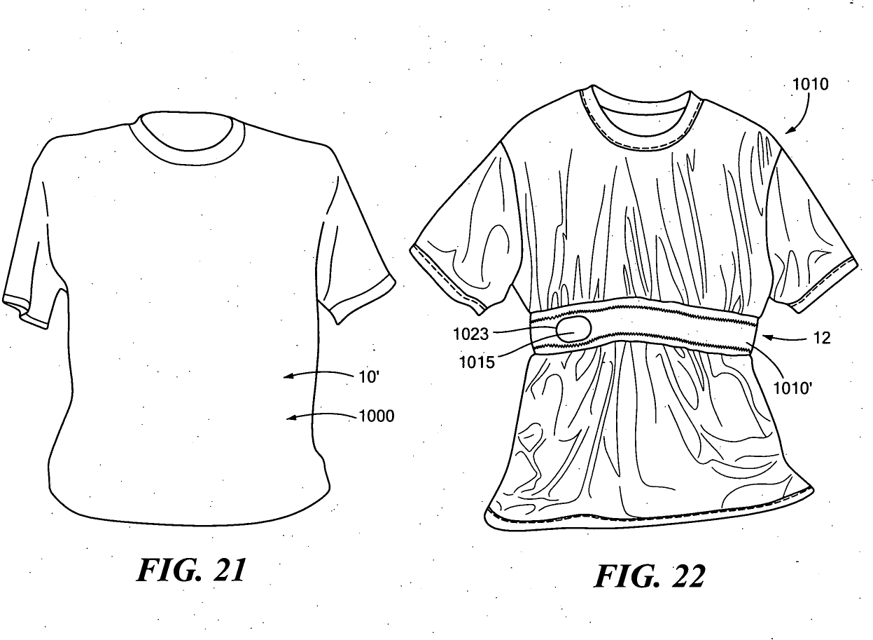

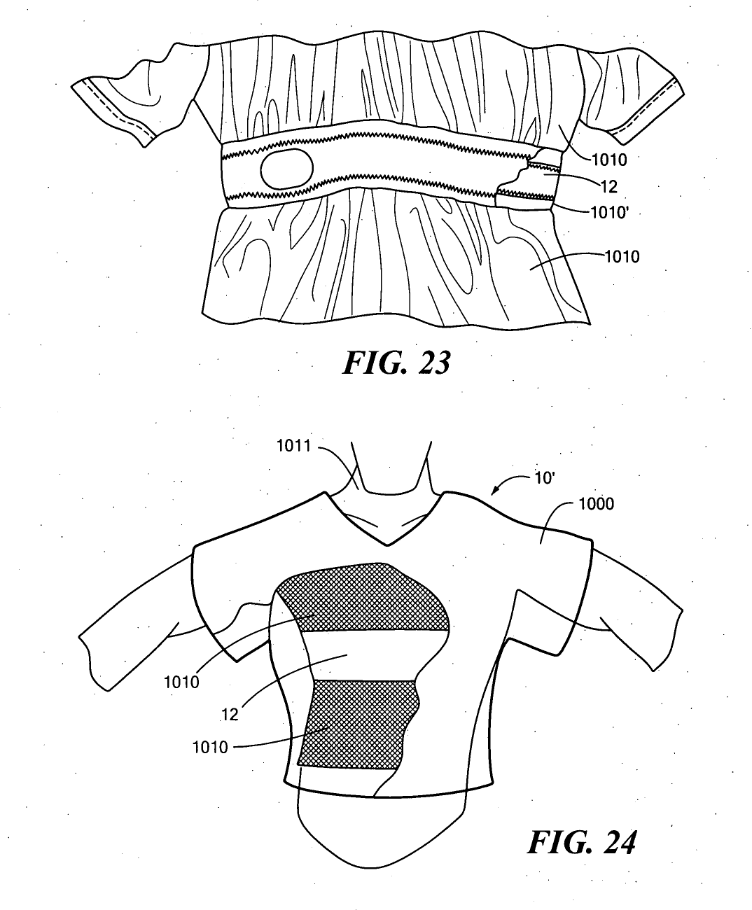

In another embodiment in accordance with the subject invention, garment or shirt 10′, FIG. 21 is made of loose-fitting material 1000 and includes inner liner or shell 1010, FIG. 22 which may be sewn or otherwise attached to the loose fitting material 1000, for example at the seams of loose-fitting material 1000, or otherwise as desired for a particular application. Loose-fitting material 1000 and inner liner 1010 may be made of known conventional garment materials or other suitable materials as desired. In this embodiment, stretchable circumferential band 12, FIG. 23 shown within inner liner portion 1010′, is sewn, bonded or attached to inner liner 1010 or inner liner portion 1010′ as shown. In this configuration, inner liner 1010 extends from each edge of loose-fitting material 1000 and to stretchable circumferential band 12. Another view of shirt 10′ is shown in FIG. 24, where there is no inner liner portion 1010′ on the side of band 12 facing away from the wearer 1011.

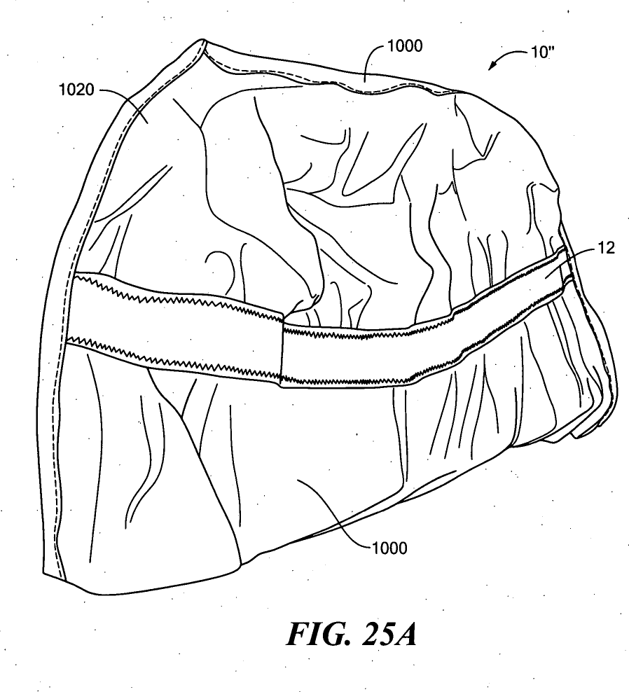

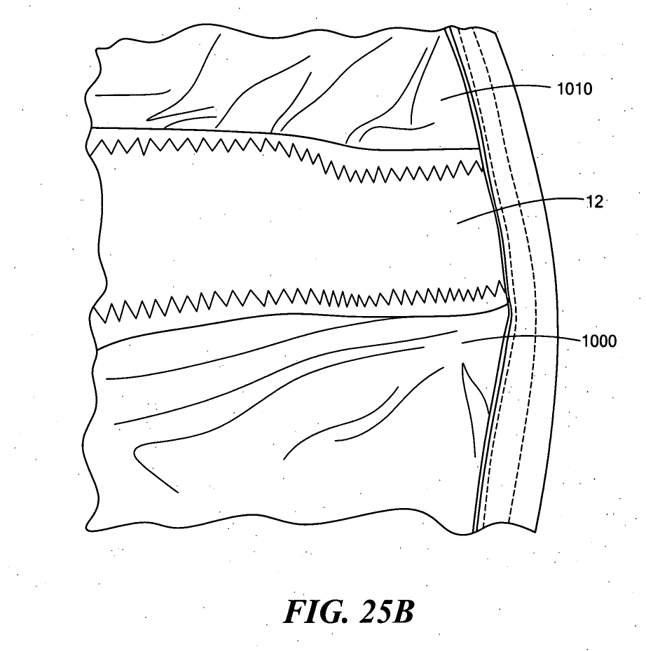

In a further embodiment, shirt 10′, FIG. 25A includes inner liner or shell 1020 which extends not from each edge of the loose-fitting material but from select edges such as the top edges of loose-fitting material 1000 (the collar and shoulder area as shown, although this is not a necessary limitation), and then to stretchable circumferential band 12. Stretchable circumferential band 12, FIG. 25B may be sewn, bonded or attached to the shirt at the inner liner 1010 and/or to loose-fitting material 1000.

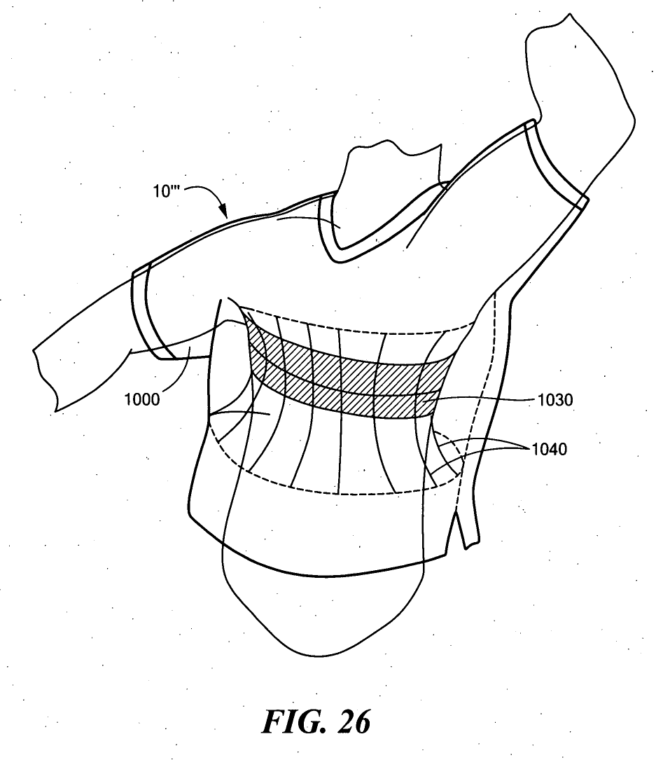

In still another embodiment, shirt 10′″, FIG. 26, includes inner liner 1030 attached to or surrounding the stretchable circumferential band with no liner material extending to the edges of loose-fitting material 1000. Instead, liner 1030, FIG. 26 attaches to loose-fitting material 1000 via connecting material segments 1040, such as POLARTEC® POWER STRETCH® material or similar fabric, stretchable or non-stretchable, although any suitable connecting material segments may be utilized. As shown, liner 1030 is on each side of stretchable circumferential band 12.

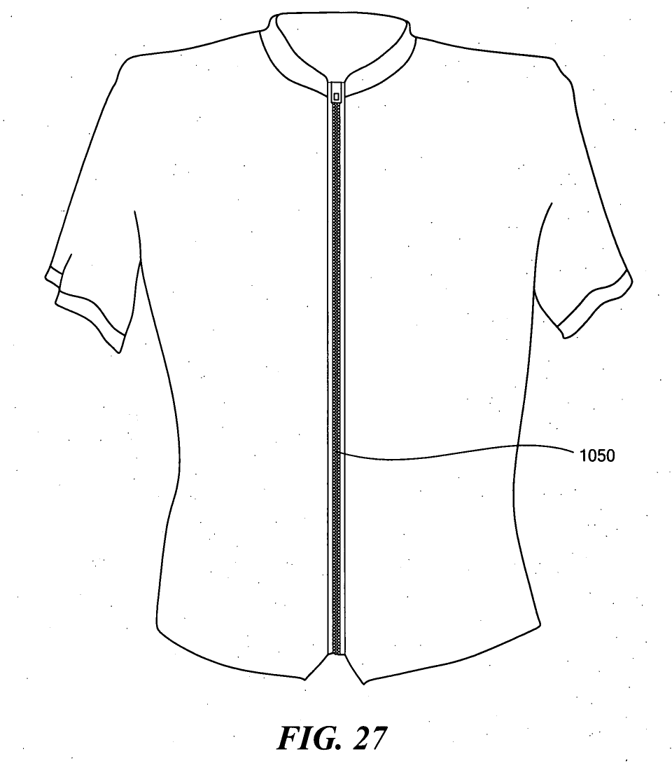

As is apparent from the descriptions above, in any of shirts 10′, 10″, or 10′″, the liner may be on both sides of band 12, or only on one side of band 12, for example, on the side of the band facing the wearer, or not on either side of the band but attached to the band at either the top and/or bottom of the band. Band 12 may also be included within the inner liner, such as when the inner liner or inner liner material surrounds the band. Typically, where the liner is included on the side of the band facing the wearer, the liner will include cutouts 1023 therein as appropriate, FIG. 22, for sensors such as sensor 1015 to make direct contact with the wearer. In the embodiments of FIGS. 21-26, the circumference or size of loose-fitting material 1000 will typically be larger than the length of band 12, and in one variation, in order to hold the band and the sensors attached to the band in more intimate contact with the wearer's body, shirts 10′, 10″, or 10′″ may include zipper or other means 1050 for closing the shirt, FIG. 27, such as buttons, hook and loop fasteners or the like, which when closed or zipped, closes the shirt and assists in tightening band 12 against the body of the wearer while loose-fitting material 1000 remains away from the wearer's body.

Accordingly, shirts 10′, 10″ and 10′″ offer alternatives to a tighter-fitting shirt, and for the most part the physiological monitoring portion of shirts 10′, 10″ and 10′″ are not visible from the viewpoint of an outside observer. Not having to wear a skin tight garment is a particular advantage for those who would not normally wear a tight garment. Additionally, a zipper-front version allows for much easier donning of the garment for the elderly, the obese, etc., while still allowing for the snug-fitting band which is necessary for the proper function of the device.

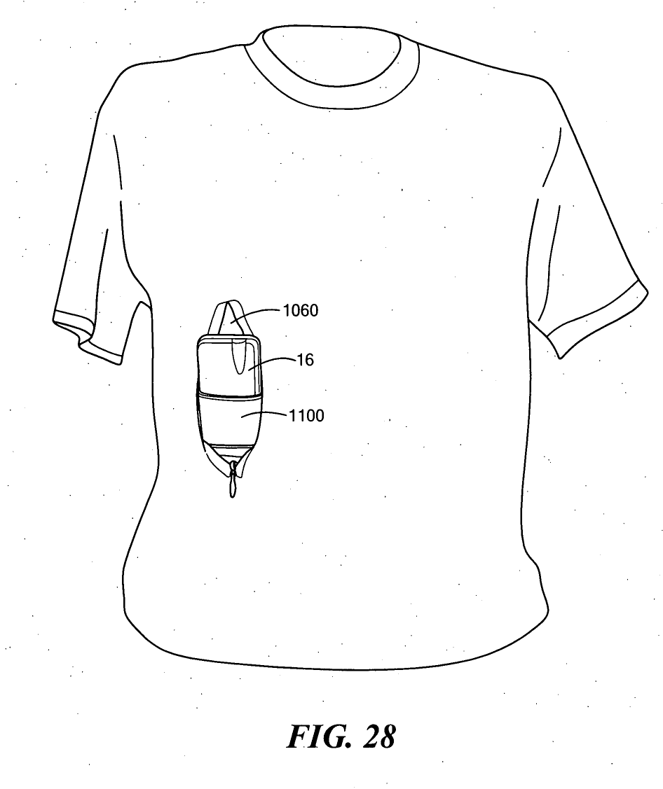

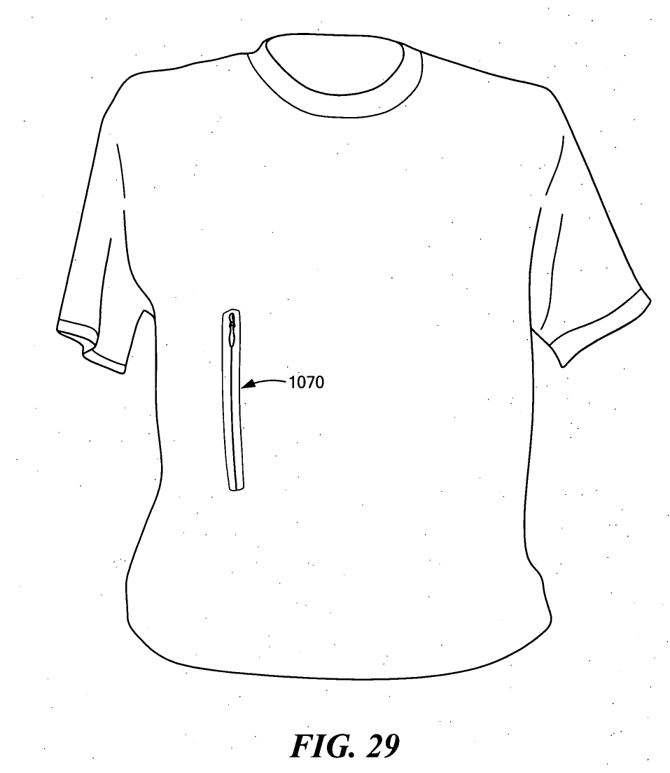

In other respects, however, shirts 10′, 10″, and 10′″ are configured, function, operate and include features similar to shirt 10 discussed above, and thus provide the same advantages provided by shirt 10. In this regard, accommodations are typically made, for example, adding opening 1060, FIG. 28 to allow for releasable attachment of electronics module 16, FIG. 29 to the shirt, particularly to the shirt inner liner. As discussed above, it will be understood that the signal transmission conductors from the sensors and the respiration detector subsystem in the stretchable circumferential band connect to the electronics module. The example shown includes optional pocket 1100 in the liner for module 16. Typically, opening 1060 is closable by zipper or other means for closing 1070, such as buttons or hook and loop fasteners, as shown in FIG. 29.

The result in any embodiment is a new physiological monitoring system, typically garment based, which is comfortable to wear, easy to use, and easy to clean. Preparation prior to and/or after donning the garment is not required. Preferably, there are no wires which must be connected or routed nor is the user required to secure electrodes to his body or to use any conductive gels. The garment whether a shirt or other article of clothing is aesthetically pleasing and does not interfere with the activities of or duties carrier out by the wearer. The shirt area at the stretchable circumferential band holds sensors in intimate contact with the body, for increased accuracy and the electronics module located away from the sensors further improves accuracy.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

What is claimed is:

1. A physiological status monitoring system comprising:
a shirt;
a stretchable circumferential band attached to the shirt, the stretchable band including:
a respiration detector subsystem integral with the band, and
signal transmission conductors also integral with the band and in a flexible configuration;
one or more sensors on the band each electrically connected to a signal transmission conductor, at least one sensor having an electrode exposed inside the shirt;
a cover over the band and/or the one or more sensors;
a connection subsystem on the band electrically connected to said respiration detector subsystem and said signal transmission conductors and including signal traces therefrom to a first connector accessible from outside the shirt;
an electronics module releasably attached to the shirt including a second connector which mates with the first connector, the electronics module including:
a processing system for processing signals from the respiration detector subsystem and the signal transmission conductors, and
a transmitter for wirelessly transmitting the processed signals; and
a remote display unit including:
a receiver which receives said transmitted processed signals,
a display, and
a processing system for displaying said received processed signals on said display.

2. The system of claim 1 in which the electronics module includes a receiver for receiving communications from the remote display unit.

3. The system of claim 1 in which said shirt is made of Lycra material.

4. The system of claim 1 in which the shirt includes fabric having fibers of variable loft and thickness.

5. The system of claim 1 in which said respiration detector subsystem includes in-plane circumferential conductors integral with said stretchable band.

6. The system of claim 5 in which the in-plane circumferential conductors are in a sinusoidal configuration.

7. The system of claim 6 including a pair of adjacent in-plane nested circumferential conductors integral with said stretchable band.

8. The system of claim 7 in which the electronics module includes a circuit which detects changes in capacitance as the adjacent nested circumferential conductors move away from and towards each other as the stretchable band expands and contracts.

9. The system of claim 5 in which the electronics module includes a circuit which detects changes in inductance as the circumferential conductors move as the stretchable band expands and contracts.

10. The system of claim 1 in which the signal transmission conductors are sinusoidal and circumferential.

11. The system of claim 1 in which the signal transmission conductors are sinusoidal.

12. The system of claim 1 in which the at least one sensor exposed electrode is made of conductive fabric.

13. The system of claim 12 in which there are one or more foam layers behind the conductive fabric to promote contact of the conductive fabric with skin.

14. The system of claim 13 further including a fabric over and behind the one or more foam layers and attached to the stretchable band and the shirt.

15. The system of claim 13 in which the one or more foam layers are water impenetrable to promote the conductivity of the conductive fabric.

16. The system of claim 12 further including a conductive fastener through the conductive fabric and connected to a conductor coupled to a signal transmission conductor in the band.

17. The system of claim 1 in which one said sensor includes a thermistor coupled to the band and having a conductor coupled to a signal transmission conductor.

18. The system of claim 17 in which a side of the thermistor not in contact with a wearer is insulated.

19. The system of claim 1 in which the cover is an outer cover.

20. The system of claim 19 in which the outer cover is made of fabric.

21. The system of claim 20 in which said outer cover fabric is the same as the shirt fabric.

22. The system of claim 1 in which the connection subsystem is an insulation displacement connector.

23. The system of claim 1 in which the connection subsystem is a circuit board.

24. The system of claim 23 in which said circuit board is rigid.

25. The system of claim 23 in which said circuit board is a flex circuit.

26. The system of claim 1 in which said first connector is a Lemo connector.

27. The system of claim 1 in which said first connector is a pin connector.

28. The system of claim 1 in which said connection subsystem is encapsulated by a sealant.

29. The system of claim 1 in which said transmitter is configured according to the Bluetooth standard and the remote display unit is a hand held electronic device.

30. The system of claim 29 in which said hand held device is a personal digital assistant.

31. The system of claim 1 further including a portal accessible over a network responsive to the remote display unit to display and log said processed signals.

32. The system of claim 31 in which said portal is a website.

33. The system of claim 1 in which said electronics module further includes a position detection subsystem.

34. The system of claim 33 in which the position detection subsystem is a GPS subsystem.

35. The system of claim 1 further including a position detection subsystem separate from but in communication with said electronics module.

36. The system of claim 1 in which said electronics module further includes a motion detector.

37. The system of claim 36 in which said motion detector is an accelerometer.

38. The system of claim 1 in which said electronics module includes a display for displaying said processed signals.

39. The system of claim 1 further including one of a hook and loop fastener on the shirt and the other of the hook and loop fastener on the electronics module for releasably attaching the electronics module to the shirt.

40. The system of claim 1 further including snap connectors for releasably attaching the electronics module to the shirt:

41. The system of claim 1 further including magnetic connectors for releasably attaching the electronics module to the shirt.

42. The system of claim 41 in which the magnetic connectors provide electrical connectivity between the shirt and the electronics module.

43. The system of claim 1 in which the processing system of the electronics module or the remote display unit includes a rules set including a rule which signals from the respiration detector subsystem are not transmitted if they indicate a breathing rate higher than possible by a subject wearing the shirt.

44. The system of claim 1 in which the one or more sensors on the band includes electrocardiogram sensors located on opposite sides of the band and positioned to form a line across a wearer's heart.

45. The system of claim 1 in which the shirt is made of loose fitting material and includes an inner liner attached to the loose fitting material.

46. The system of claim 45 in which the inner liner extends to each edge of the loose fitting material.

47. The system of claim 45 in which the inner liner extends from select edges of the loose fitting material to the stretchable circumferential band.

48. The system of claim 45 in which the inner liner is attached to the loose fitting material by connecting material segments.

49. The system of claim 45 in which the loose fitting material includes at least one opening therein for releasable attachment of the electronics module to the shirt.

50. The system of claim 45 in which the stretchable circumferential band is included in the inner liner.

51. A physiological status monitoring system comprising:
a garment;
a stretchable band attached to or integral with the garment, the stretchable band including:
conductors integral with the band forming a respiration detector, and
at least one signal transmission conductor also integral with the band;
at least one sensor electrically connected to the signal transmission conductor; and
an electronics module responsive to the conductors and the signal transmission conductor, the electronics module including:
a circuit which detects changes in impedance as geometry of the conductors changes as the stretchable band expands and contracts, and
a transmitter responsive to the circuit and to signals transmitted from said sensor for wirelessly transmitting respiration and sensor signals.

52. The system of claim 51 further including an outside cover over the band.

53. The system of claim 51 further including a connection subsystem on the band electrically connected to said conductors and to said signal transmission conductor and including signal traces therefrom to a first connector accessible from outside the garment.

54. The system of claim 53 in which the electronics module includes a second connector which mates with the first connector.

55. The system of claim 51 in which the electronics module further includes a processing system for processing signals from the circuit and the signal transmission conductor.

56. The system of claim 51 in which the conductors are in a sinusoidal configuration.

57. The system of claim 56 in which the at least one signal transmission conductor is in a sinusoidal configuration.

58. The system of claim 51 in which the conductors include a pair of adjacent nested conductors.

59. The system of claim 55 further including a remote display unit comprising:
a receiver which receives signals from the transmitter,
a display, and
a processing system for displaying said received signals on said display.

60. The system of claim 59 in which the electronics module includes a receiver which is responsive to the remote display unit and to signals transmitted from said remote display unit for receiving communications from the remote display unit.

61. The system of claim 51 in which said garment is made of Lycra material.

62. The system of claim 51 in which the garment includes fabric having fibers of variable loft and thickness.

63. The system of claim 51 in which said band is circumferential and said conductors are in-plane.

64. The system of claim 51 in which the at least one sensor includes an exposed electrode made of conductive fabric.

65. The system of claim 64 in which there are one or more foam layers behind the conductive fabric to promote contact of the conductive fabric with skin.

66. The system of claim 65 further including fabric over and behind the one or more foam layers and attached to the stretchable band.

67. The system of claim 66 in which the one or more foam layers are water impenetrable to promote the conductivity of the conductive fabric.

68. The system of claim 64 further including a conductive fastener through the conductive fabric and connected to a conductor coupled to a signal transmission conductor in the band.

69. The system of claim 51 in which one said sensor includes a thermistor coupled to the band and having a conductor coupled to a signal transmission conductor.

70. The system of claim 69 in which a side of the thermistor not in contact with a wearer is insulated.

71. The system of claim 53 in which the connection subsystem is an insulated displacement connector.

72. The system of claim 53 in which the connection subsystem is a circuit board.

73. The system of claim 72 in which said circuit board is rigid.

74. The system of claim 72 in which said circuit board is a flex circuit.

75. The system of claim 53 in which said first connector is a Lemo connector.

76. The system of claim 53 in which said first connector is a pin connector.

77. The system of claim 51 in which said electronics module further includes a position detection subsystem.

78. The system of claim 77 in which the position detection subsystem is a GPS subsystem.

79. The system of claim 51 further including a position detection subsystem separate from but in communication with said electronics module.

80. The system of claim 51 in which said electronics module further includes a motion detector.

81. The system of claim 80 in which said motion detector is an accelerometer.

82. The system of claim 51 in which said electronics module includes a display for displaying said respiration and sensor signals.

83. The system of claim 51 further including one of a hook and loop fastener on the garment and the other of the hook and loop fastener on the electronics module for releasably attaching the electronics module to the garment.

84. The system of claim 51 further including snap connectors for releasably attaching the electronics module to the garment.

85. The system of claim 51 further including magnetic connectors for releasably attaching the electronics module to the garment.

86. The system of claim 85 in which the magnetic connectors provide electrical connectivity between the garment and the electronics module.

87. The system of claim 51 further including a processing system including a rules set including a rule in which signals from the respiration detector are not transmitted if they indicate a breathing rate higher than possible by a subject wearing the garment.

88. The system of claim 51 further including electrocardiogram sensors on the stretchable band located on opposite sides of the band and positioned to form a line across a wearer's heart.

89. The system of claim 51 in which the garment is made of loose fitting material and includes an inner liner attached to the loose fitting material.

90. The system of claim 89 in which the inner liner extends to each edge of the loose fitting material.

91. The system of claim 89 in which the inner liner extends from select edges of the loose fitting material to the stretchable circumferential band.

92. The system of claim 88 in which the inner liner is attached to the loose fitting material by connecting material segments.

93. The system of claim 88 in which the loose fitting material includes at least one opening therein for releasable attachment of the electronics module to the garment.

94. The system of claim 89 in which the stretchable band is included in the inner liner.

95. A physiological status monitoring system comprising:
a stretchable circumferential band including:
a respiration detector subsystem integral with the band, and
signal transmission conductors also integral with the band and in a flexible configuration;
one or more sensors on the band and electrically connected to a signal transmission conductor, at least one sensor having an exposed electrode;
a connection subsystem on the band electrically connected to said respiration detector subsystem and the signal transmission conductors and including signal traces therefrom to a first connector; and
an electronics module with a second connector which mates with the first connector, the electronics module including:
a processing system for processing signals from the respiration detector subsystem and the signal transmission conductors, and
a transmitter for wirelessly transmitting the processed signals.
